(19) United States
(12) Patent Application Publication
(10) Pub. No.: US 2014/0236012 A1
(43) Pub. Date: Aug. 21, 2014

(54) ULTRASOUND DIAGNOSIS APPARATUS

(71) Applicants: Kabushiki Kaisha Toshiba, Minato-ku (JP); Toshiba Medical Systems Corporation, Otawara-shi (JP)

(72) Inventors: Shouichi Nakauchi, Nasushiobara-shi (JP); Takashi Koyakumaru, Otawara-shi (JP); Takashi Masuda, Nasushiobara-shi (JP); Takeshi Obara, Otawara-shi (JP); Naoki Yoneyama, Otawara-shi (JP)

(73) Assignees: Kabushiki Kaisha Toshiba, Minato-ku, Tokyo (JP); Toshiba Medical Systems Corporation, Otawara-shi, Tochigi (JP)

(21) Appl. No.: 14/347,916

(22) PCT Filed: Aug. 15, 2013
(86) PCT No.: PCT/JP2013/071945

§ 371 Date: Mar. 27, 2014

(30) Foreign Application Priority Data
Aug. 17, 2012 (JP) 2012-180763

(51) Int. Cl. A61B 8/00; A61B 8/06; A61B 8/14

(52) U.S. Cl. CPC A61B 8/4254; A61B 8/14; A61B 8/461; A61B 8/06
USPC 600/443

(57) ABSTRACT

Ultrasound diagnosis apparatus is provided which easily recreates test conditions same as the past, comprising scanner, signal processor, display controller, storage, detector, collator, and controller. Scanner scans a subject with ultrasound waves through ultrasound probe. Storage previously stores first relevant information in which operation of the scanner, the signal processor, or the display controller in past test is related to a first relative position which is the relative position of ultrasound probe for the subject in the same past test. Detector detects a second relative position which is the present relative position of ultrasound probe for the subject. Collator collates the first relative position and the second relative position detected by the detector. Controller controls the scanner, the signal processor or the display controller based on the operation related to the first relative position, when the first relative position and the second relative position are matched in collation.

### TECHNICAL FIELD

The embodiments of the present invention relate to an ultrasound diagnosis apparatus.

### BACKGROUND ART

Ultrasound diagnosis apparatuses obtain biological information of a subject by transmitting ultrasound waves to the subject using an ultrasound probe and receiving the reflected waves thereof. The ultrasound diagnosis apparatuses can perform tests repeatedly, since the apparatuses have a high level of safety. Further, since the scale of their system is smaller than the scales of the other diagnosis apparatuses, such as X-ray diagnosis apparatuses, X-ray CT (Computed Tomography) apparatuses, and MRI (Magnetic Resonance Imaging) apparatuses, it is easy to use the ultrasound diagnosis apparatuses for performing tests by a bedside, for example. Furthermore, the ultrasound diagnosis apparatuses do not expose X-rays, therefore, can be used for obstetrics, home medical care, and the like.

Operation content of the ultrasound diagnosis apparatus includes content of many kinds, for example, depth of focus, frequency, parallel reception number, contrast, gain, operation of application software for analyzing biological information obtained, and the like. Further, the operation content and the relative position of the ultrasound probe for the subject are updated in real time during the test in accordance with the test circumstances. Therefore, in order to test under the same conditions as the one in the past test, for example, such a case that a test is required under the same conditions again during the test, and a case that a test is performed for follow-up observations, it is necessary to recreate the operation content of the ultrasound diagnosis apparatus and the relative position of the ultrasound probe for the subject.

### PRIOR ART DOCUMENT

### Patent Document

[Patent Document 1] Japanese Unexamined Patent Application Publication No. 11-206767

### SUMMARY OF THE INVENTION

### Problems to be Solved by the Invention

As described, it is necessary to recreate the operation content of the ultrasound diagnosis apparatus and the relative position of the ultrasound probe for the subject, in order to test under the same conditions as the one in the past (in the present invention, the operational conditions and the relative position are referred to as test conditions). However, in order to recreate the operation content in the past, it is necessary to set many kinds of operation content according to the past operation content, and thus the setting work has been complicated. It is also necessary to position the ultrasound probe at the past relative position for the subject to recreate the relative position. It has been difficult however to recreate the relative position, since the ultrasound probe is manually operated by an operator.

The present invention is intended to provide an ultrasound diagnosis apparatus which is able to easily recreate test conditions which are the same as in the past.

### Means of Solving the Problems

The ultrasound diagnosis apparatus in the present embodiments comprises a scanner, a signal processor, a display controller, a storage, a detector, a collator, and a controller. The scanner scans a subject with ultrasound waves through an ultrasound probe. The signal processor implements signal processing on signals from the scanner. The display controller causes a display to display images, based on the output from the signal processor. The storage previously stores first relevant information in which operation content of at least one of the scanner, the signal processor, and the display controller in the past test is related to a first relative position which is the relative position of the ultrasound probe for the subject in the same past test . The detector detects a second relative position which is the present relative position of the ultrasound probe for the subject. The collator collates the first relative position represented in the first relevant information and the second relative position detected by the detector. The controller controls at least one of the scanner, the signal processor, and the display controller, based on the operation content related to the first relative position, when the first relative position and the second relative position are matched in the collation by the collator.

### BRIEF DESCRIPTION OF THE DRAWINGS

FIG. 1 is a block diagram illustrating a configuration of an ultrasound diagnosis apparatus of an embodiment.

FIG. 2 is a schematic view illustrating an outline of the ultrasound diagnosis apparatus of the embodiment.

FIG. 3 is a schematic view illustrating an outline of the ultrasound diagnosis apparatus of the embodiment.

FIG. 4 is a flowchart illustrating operation of the ultrasound diagnosis apparatus of the embodiment.

FIG. 5 is a block diagram illustrating a configuration of the ultrasound diagnosis apparatus of an embodiment.

FIG. 6 is a block diagram illustrating a configuration of the ultrasound diagnosis apparatus of an embodiment.

### MODES FOR CARRYING OUT THE INVENTION

Hereinafter, it is described the ultrasound diagnosis apparatus of the embodiments with reference to the drawings.

### First Embodiment

### Configuration

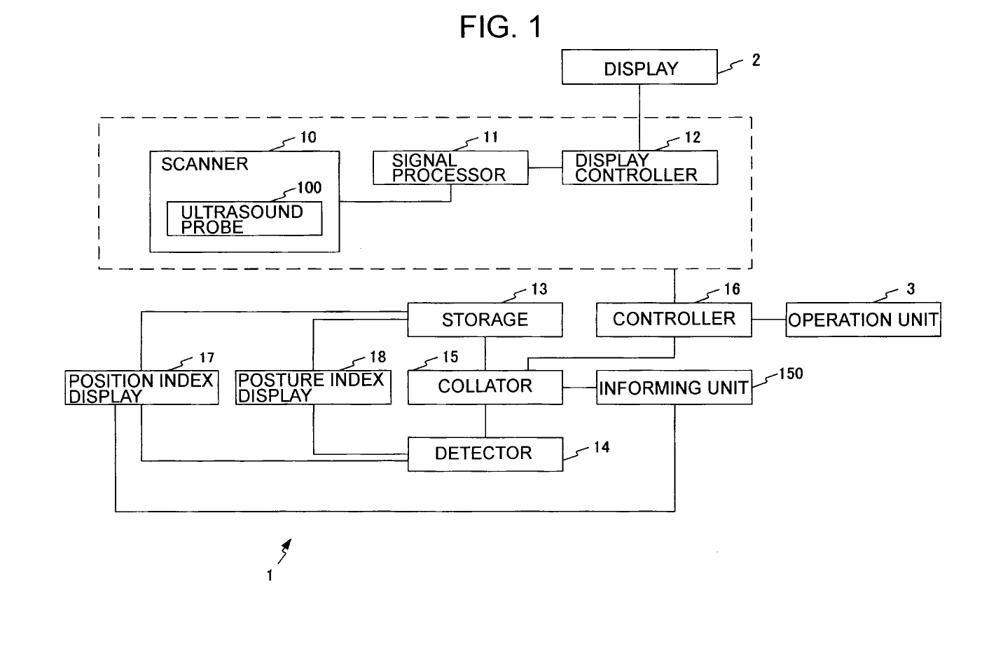

FIG. 1 is a block diagram illustrating a configuration of an ultrasound diagnosis apparatus 1 of the embodiment. The ultrasound diagnosis apparatus 1 comprises a scanner 10, a signal processor 11, a display controller 12, a storage 13, a detector 14, a collator 15, a controller 16, a position index display 17, a posture index display 18, an informing unit 150, a display 2, and an operation unit 3. The display 2 and the operation unit 3 are arranged either inside or outside of the ultrasound diagnosis apparatus 1.

### (Scanner 10)

The scanner 10 scans a subject with ultrasound waves through an ultrasound probe 100. For the ultrasound probe 100, for example, a one-dimensional array probe in which a plurality of ultrasound transducers are arranged in a row in the scanning direction, or a two-dimensional array probe in which a plurality of ultrasound transducers are arranged two-dimensionally is used. A mechanical one-dimensional array probe in which a plurality of ultrasound transducers arranged in a row in the scanning direction is swung in the swinging direction orthogonal to the scanning direction may also be used. The ultrasound probe 100 transmits the ultrasound waves to the subject, and receives the reflected waves from the subject as echo signals.

The scanner 10 supplies electric signals to the ultrasound probe 100, and causes the probe 100 to transmit beamformed (that is, transmission beamformed) ultrasound waves to a predetermined focus point. Further, the scanner 10 receives the echo signals the ultrasound probe 100 received. The scanner 10 implements delay processing on the echo signals to convert the analog echo signals into phased (that is, reception beamformed) digital data.

The scanner 10 comprises, for example, a not-shown preamplifier circuit, an A/D converter, a reception delay circuit, and an adder. For each reception channel, the preamplifier circuit amplifies the echo signals output from each of the ultrasound transducers of the ultrasound probe 100. The A/D converter converts amplified echo signals into digital signals. The reception delay circuit gives delay time required for determining reception directivity to the echo signals which has been converted into the digital signals. The adder adds the echo signals to which the delay time has been given. The reflection component from the direction according to the reception directivity is emphasized by the addition.

(Signal processor 11)

The signal processor 11 implements signal processing on the signals from the scanner 10. For example, the signal processor 11 comprises a B-mode processor. The B-mode processor receives the signals from the scanner 10, and images the amplitude information of the signals. Specifically, the B-mode processor implements band pass filter processing on the signals, and then performs envelop detection of the signals to implements compression processing on the detected data by logarithmic conversion.

The signal processor 11 may comprise a CFM (Color Flow Mapping) processor. The CFM processor images blood flow information. The blood flow information includes information, such as velocity, distribution, and power, and is obtained as binarization information.

The signal processor 11 may comprise a Doppler processor. The Doppler processor performs phase detection on the signals to take out the Doppler shift frequency component therefrom, and implements FFT (Fast Fourier Transform) processing thereon to generate a Doppler frequency distribution representing the blood flow velocity.

The signal processor 11 generates ultrasound image data based on the signals which have been subjected to the signal processing (ultrasound raster data). The signal processor 11, for example, comprises a DSC (Digital Scan Converter). The signal processor 11 converts the signals represented by a signal stream of the scanning line, on which the signal processing has been implemented, into image data represented by orthogonal coordinate system (scan conversion processing). For example, the signal processor 11 generates B-mode image data representing the tissue form of a subject P by implementing the scan conversion processing on the signals subjected to the signal processing by the B-mode processor. The signal processor 11 outputs the ultrasound image data to the display controller 12. Further, the content of the signal processing performed by the signal processor may be designated by using predetermined analysis application software.

(Display controller 12)

The display controller 12 causes the display 2 to display images based on the output from the signal processor 11. That is, the display controller 12 receives the ultrasound image data from the signal processor 11 to cause the display 2 to display the ultrasound images based on the ultrasound image data. Further, the display controller 12 may display the signal processing result resulted by the signal processor 11 using predetermined analysis application software.

### (Storage 13)

The storage 13 previously stores first relevant information in which operation content of at least one of the scanner 10, the signal processor 11, and the display controller 12 in a past test is related to a first relative position which is the relative position of the ultrasound probe 100 for the subject P in the same past test . The first relative position represents the position of the ultrasound probe 100 for the characteristic point of the subject P. The operation content includes scanning conditions for the ultrasound waves from the scanner 10, signal processing conditions for the signal processor 11, and display conditions for the images to be displayed by the display controller 12. For example, the scanning conditions may include conditions, such as depth of focus, a parallel-simultaneous reception number, and the like. Further, the signal processing conditions may include signal processing conditions corresponding to operating modes, such as B-mode, Doppler mode, and the like. Furthermore, the display conditions may include contrast and brightness of the image, ON/OFF of a freezing display function of the image, and the like. In addition, the storage 13 may previously store the first relevant information including test item information representing test items in the operation conditions.

Further, the first relative position in the first relevant information previously stored in the storage 13 includes the relative positions of plural parts of the ultrasound probe 100 for the subject P. For example, in the ultrasound probe 100, the first relative position includes the relative positions of three parts including the tip end part (a part of the ultrasound transducer side), the base end part (the opposite side of the tip end part), and the side face part (may be any arbitrary part distinguishable from the tip end part and the base end part) for the subject P, as one pair. That is, in the first relevant information, operational information is related to one pair of relative positions of the plural parts of the ultrasound probe 100. The relative positions of the plural parts represent an angle of the ultrasound probe 100. Further, the storage 13 may store the form of the ultrasound probe 100.

The storage 13 may also previously store posture information representing the posture of the subject P in the past test. At this time, the storage 13 stores the relative positions of the other characteristic points for a predetermined point among a plurality of characteristic points (for example, characteristic points such as neck region, pectoral region, abdominal region, right shoulder, and left shoulder) of the subject P, as the posture information. Further, the posture information may include contour information representing the contours of the body surface of the subject P.

The storage 13 may also previously store second relevant information in which order information representing the moving order of the ultrasound probe in the past test is related to the first relative position . For example, in a test performed while the ultrasound probe 100 is moved, the storage 13 may relate a plurality of positions representing the moving process (positions to scan the subject) with the order thereof to store.

### (Detector 14)

The detector 14 detects a second relative position which is the relative position of the ultrasound probe 100 for the subject P in the present test. For example, the detector 14 may detect the second relative position by detecting predetermined parts of the subject P and the ultrasound probe 100 using a magnetic sensor. The detector 14 may also detect the second relative position by detecting the positions of the subject P and the ultrasound probe 100 using an optical sensor and triangulation. Further, the detector 14 may detect the relative positions of plural parts of the ultrasound probe 100 for the subject P as the second relative position. The relative positions for the plural parts represent an angle of the ultrasound probe 100. The detector 14 may also detect the posture of the subject P. The posture is a physical posture and form of the subject P. The detector 14 may relate part information and/or the detection conditions (such as sensor resolution) representing the detected predetermined parts of the subject P and the ultrasound probe 100 with the second relative position to output.

### (Collator 15)

The collator 15 collates the first relative position represented in the first relevant information previously stored in the storage 13 and the second relative position detected by the detector 14. At this time, the collator 15 receives the first relative position from the storage 13, and the second relative position, as a comparable object, from the detector 14. The collator 15 outputs a collation result whether the first relative position and the second relative position are matched or not by the collation. In the collation, the collator 15 outputs the collation result such that the first relative position and the second relative position are matched, when the second relative position exists within a predetermined range from the first relative position. The predetermined range is, for example, previously stored in the collator 15 as corresponding information in which the test items and the detection conditions for the detector 14 correspond to the predetermined range. Also, the operator may designate a desired range as the predetermined range. When the first relative position and the second relative position are not matched, that is, the second relative position does not exist within the predetermined range from the first relative position, the collator 15 may output the direction and/or the distance from the second relative position to the first relative position included in the collation result. The collator 15 may also collate the first relative positions and the second relative positions for the plural parts of the ultrasound probe 100.

### (Controller 16)

When the first relative position and the second relative position are matched in the collation by the collator 15, the controller 16 controls at least one of the scanner 10, the signal processor 11, and the display controller 12, based on the operation content related to the first relative position. That is, during the test, when the ultrasound probe 100 is positioned at the same relative position as the first relative position, the operation content (that is, the operation content in the past test) related to the first relative position is recreated by the controller 16. For example, the controller 16 may recreate at least one of the scanning conditions (frequency, depth of focus) of the ultrasound waves from the scanner 10, the signal processing conditions (B-mode, Doppler mode, and analysis conditions for the analysis application software) for the signal processor 11, and the display conditions (contrast of images, and display conditions for the analysis result by the analysis application software) for the display controller 12. Further, when the first relative position and the second relative position are not matched, the controller 16 controls each part in accordance with the predetermined operation content, such as the idling operation prior to test, for example.

### (Position Index Display 17)

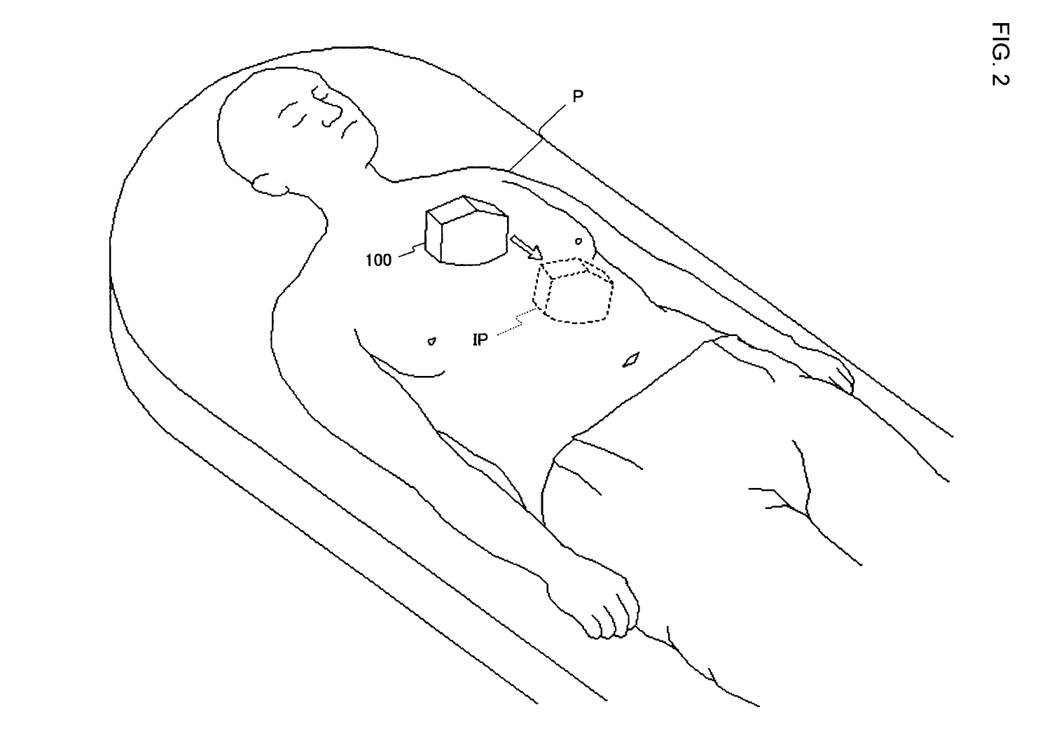

The position index display 17 displays a position index representing the first relative position, based on the first relevant information previously stored in the storage 13. FIG. 2 is a schematic view illustrating a display example of a position index IP, and represents a scene that the operator visually recognizes the subject P through the position index display. The position index display 17 implements position alignment of the characteristic points of the subject P stored in the storage 13 and the characteristic points of the subject P detected by the detector 14. The position index display 17 then displays the position index IP based on the position-aligned characteristic points and the first relevant information stored in the storage 13.

Further, the position index display 17 may have a configuration such that the display 17 has a transparent medium, displays the position index IP on a part of the medium, and is wearable by the operator. At this time, the position index display 17 may be configured as in a form of an eyewear shape, for example.

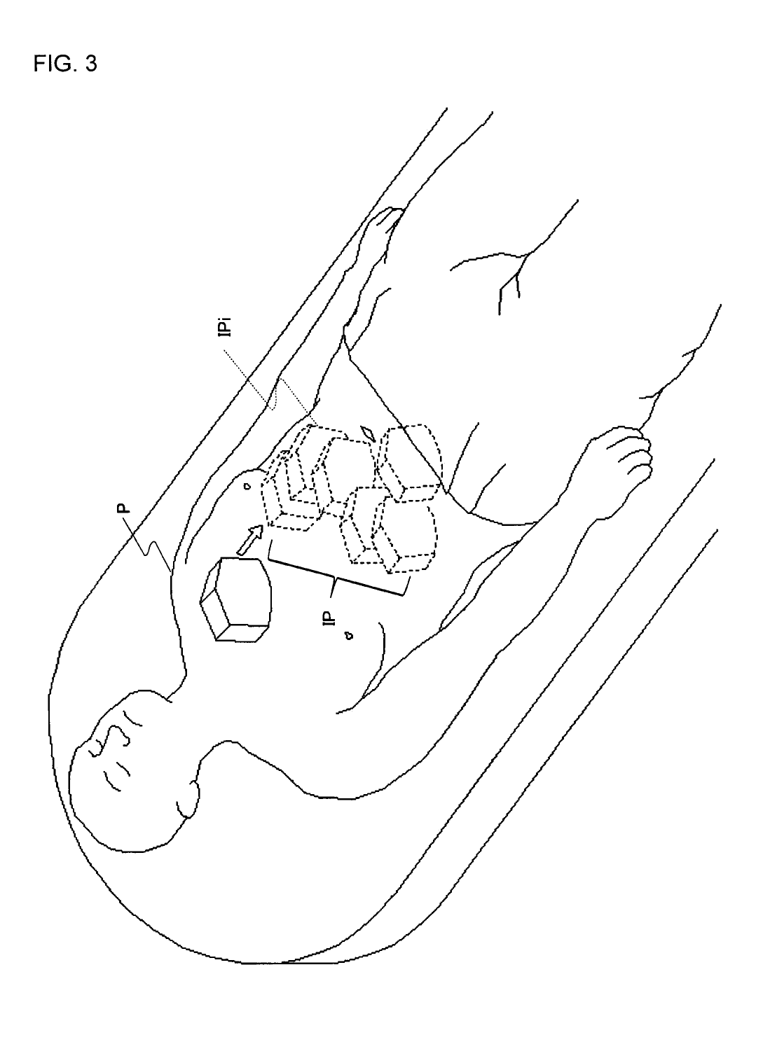

Furthermore, the position index display 17 may display a plurality of position index IPs based on the first relevant information and the second relevant information. FIG. 3 is a schematic view illustrating an example that a plurality of position index IPs are displayed. The plurality of position index IPs illustrated in FIG. 3 are position index IPi (i=1 to n; n is a number of the position index IP displayed). The position index display 17 also displays one of the plurality of position index IPs in a display mode different from the others, for example. The position index IP in the different display mode may be configured to indicate a position at which the ultrasound probe 100 to be positioned at first among the plurality of position index IPs, for example. It may be configured that the display of the position index IP of which the ultrasound probe 100 is positioned among the plurality of position index IPs is sequentially terminated.

### (Posture Index Display 18)

The posture index display 18 displays the posture index imitating the posture of the subject P in the past test, based on the posture information stored in the storage 13. The posture index display 18 may also display characteristic points of the subject P during a test in response to the detection result from the detector 14. Further, the posture index display 18 may have a configuration such that the display 18 has a transparent medium, displays the posture index on a part of the medium, and is wearable by the operator. The posture index display 18 may also be configured with the position index display 17 as a whole.

### (Informing Unit 150)

The informing unit 150 informs the collation result from the collator 15. For example, as the result of the collation, when the first relative position and the second relative position are matched, the collator 15 informs the matching by, for example, beep sound, changing the color of the position index IP, or the like. When the first relative position and the second relative position are not matched, the collator 15, for example, calculates the difference between the first relative position and the second relative position, and outputs the information representing the distance and/or direction from the second relative position to the first relative position.

### (Display 2)

The display 2 displays ultrasound images. The display 2 may be configured with a display device, such as a CRT (Cathode Ray Tube), a LCD (Liquid Crystal Display), or the like. The display 2 does not necessarily need to be integrally arranged with the ultrasound diagnosis apparatus 1, but may be configured such that the display 2 is controlled by the display controller 12 via a general interface, and displays ultrasound images.

### (Operation Unit 3)

In response to an operation by the operator, the operation unit 3 inputs the signals and the information according to the content of the operation to each part of the apparatus. The operation unit 3 may be configured with a keyboard, a mouse, a touch panel, and the like. Further, the operation unit 3 does not necessarily need to be integrally arranged with the ultrasound diagnosis apparatus 1, but may be configured such that the operation unit 3 inputs the signals and the information to each part of the apparatus via a general interface.

### Operation

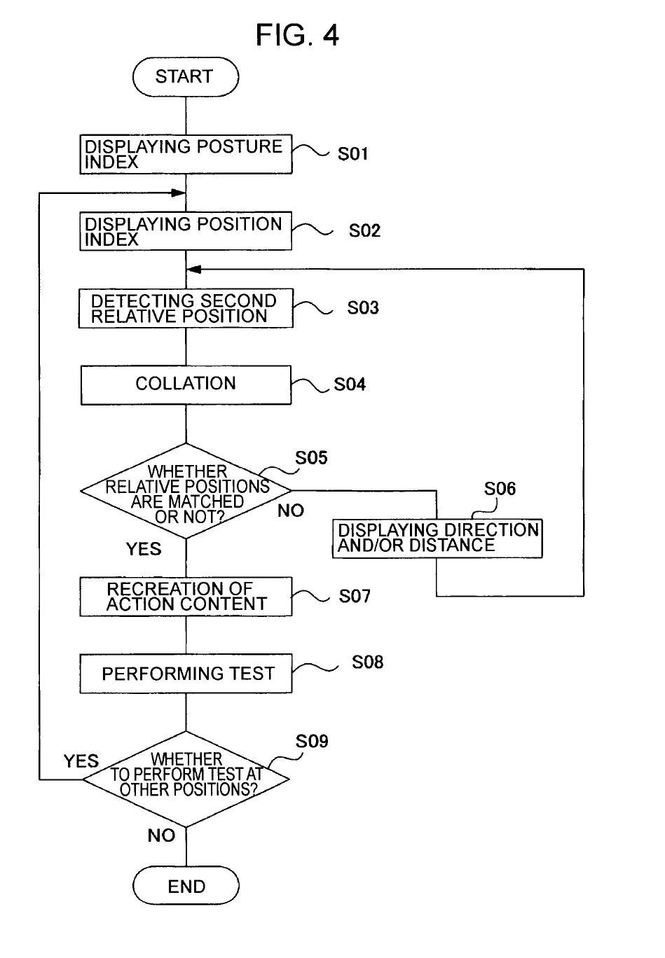

It is described operation of the ultrasound diagnosis apparatus of the embodiment. FIG. 4 is a flowchart representing the operation of the ultrasound diagnosis apparatus 1.

### (S01)

The posture index display 18 displays the posture index imitating the posture of the subject P in the past test, based on the posture information stored in the storage 13. The operator makes the posture of the subject P be matched with the posture index.

### (S02)

The position index display 17 displays the position index representing the first relative position, based on the first relevant information previously stored in the storage 13.

### (S03)

The detector 14 detects the second relative position which is the relative position of the ultrasound probe 100 for the subject P in the present test.

### (S04, S05)

The collator 15 collates the first relative position represented in the first relevant information with the second relative position detected by the detector 14. At this time, the collator 15 receives the first relative position from the storage 13, and receives the second relative position from the detector 14. In the collation, the collator 15 outputs whether the first relative position and the second relative position are matched or not as the collation result.

### (S06)

As the result of the collation by the collator 15, when the first relative position and the second relative position are not matched, the position index display 17 displays the direction and/or distance from the second relative position to the first relative position, in response to the collation result. The process is then returned to the process of step S03.

### (S07)

As the result of the collation by the collator 15, when the first relative position and the second relative position are matched, the controller 16 controls at least one of the scanner 10, the signal processor 11, and the display controller 12, based on the operation content related to the first relative position.

### (S08)

The ultrasound diagnosis apparatus 1 performs a test based on the operation content.

### (S09)

When there is a need to perform a test by positioning the ultrasound probe 100 at the other positions, such as a case when the test is performed by sequentially positioning the ultrasound probe at a plurality of positions, the process is returned to the process of step S02. When there is no need to position the ultrasound probe 100 at the other positions to test, the test is terminated. As stated above, the operation illustrated in FIG. 4 is completed.

### Operation/Effect

It is described operation and effect of the ultrasound diagnosis apparatus of the embodiment.

The ultrasound diagnosis apparatus 1 of the embodiment comprises the scanner 10, the signal processor 11, the display controller 12, the storage 13, the detector 14, the collator 15, and the controller 16. The scanner 10 scans the subject P with the ultrasound waves through the ultrasound probe 100. The signal processor 11 implements the signal processing on the signals from the scanner 10. The display controller 12 causes the display 2 to display images based on the output from the signal processor 11. The storage 13 previously stores the first relevant information in which the operation content of at least one of the scanner 10, the signal processor 11, and the display controller 12 in the past test is related to the first relative position which is the relative position of the ultrasound probe 100 for the subject P in the same past test. The detector 14 detects the second relative position which is the relative position of the ultrasound probe 100 for the subject P in the present test. The collator 15 collates the first relative position represented in the first relevant information and the second relative position detected by the detector. When the first relative position and the second relative position are matched in collation by the collator 15, the controller 16 controls at least one of the scanner 10, the signal processor 11, and the display controller 12, based on the operation content related to the first relative position. Further, the storage 13 may previously store the first relevant information including at least one of the scanning conditions of the ultrasound waves from the scanner 10, the signal processing conditions for the signal processor 11, and the display conditions of the images for the display controller 12, as the operation content. Furthermore, in the first relevant information previously stored in the storage 13, the first relative position may include the relative positions of the plural parts of the ultrasound probe 100 for the subject P, the detector 14 may detect the relative positions of the plural parts of the ultrasound probe 100 for the subject P as the second relative position, and the collator 15 may collate the first relative position and the second relative position for the plural parts of the ultrasound probe 100. In this way, when the ultrasound probe 100 is positioned at the same position as the one in the past test, the ultrasound diagnosis apparatus 1 can recreate the operation content of the past test to test. It is thereby possible to provide an ultrasound diagnosis apparatus which can easily recreate the same test conditions as the one in the past.

Also, based on the first relevant information, the ultrasound diagnosis apparatus 1 may further comprise the position index display 17 which displays the position index IP representing the first relative position. Further, the position index display 17 may have a configuration such that the display 17 has a transparent medium, and displays the position index IP on a part of the medium, and is wearable by the operator. In this way, the ultrasound diagnosis apparatus 1 is configured such that the subject P, the ultrasound probe 100, and the position index IP are visible during the test. It is therefore possible to provide an ultrasound diagnosis apparatus which can easily position the ultrasound probe 100 at the same position as the one in the past test.

Further, the storage 13 may previously store the second relevant information in which the order information representing the moving order of the ultrasound probe 100 in the past test is related to the first relative position, and the position index display 17 may display a plurality of position index IPs based on the first relevant information and the second relevant information. In this way, by displaying the plurality of the position index IPs, the ultrasound diagnosis apparatus 1 can represent the moving procedure of the ultrasound probe 100 even in a test performed during movement of the ultrasound probe 100. Thereby, it is possible to provide an ultrasound diagnosis apparatus which can easily recreate the same test conditions as the one in the past, even in a test performed during movement the ultrasound probe 100.

Furthermore, the storage 13 may previously store posture information representing the posture of the subject P in the past test, and further comprise a posture index display 18 representing the posture index imitating the posture of the subject P in the past test, based on the posture information. Also, the posture index display 18 may have a configuration such that the display 18 has a transparent medium, displays the posture index on a part of the medium, and is wearable by the operator. The posture information may also include the contour information representing the contours of the body surface of the subject P. In this way, the operator can easily recreate the posture of the subject P in the past test based on the posture index. Thereby, it is possible to provide an ultrasound diagnosis apparatus which can easily recreate the same posture of the subject P as the one in the past, and further easily recreate the same test conditions as the one in the past.

The informing unit 150 informs the collation result resulted from the collator. Thereby, the operator can easily recognize that the first relative position and the second relative position are matched. Further, when the first relative position and the second relative position are not matched, the operator can change the second relative position by referring to the information representing the distance and/or direction from the second relative position to the first relative position represented by the informing unit 150. It is therefore possible to provide an ultrasound diagnosis apparatus which can easily recreate the same test conditions as the one in the past, and easily inform that the test conditions have been recreated.

### Second Embodiment

An ultrasound diagnosis apparatus of a second embodiment is an ultrasound diagnosis apparatus which can test a subject whose somatotype (body shape) is different from the subject in the past test, based on the operation content corresponding to the past test.

### Configuration

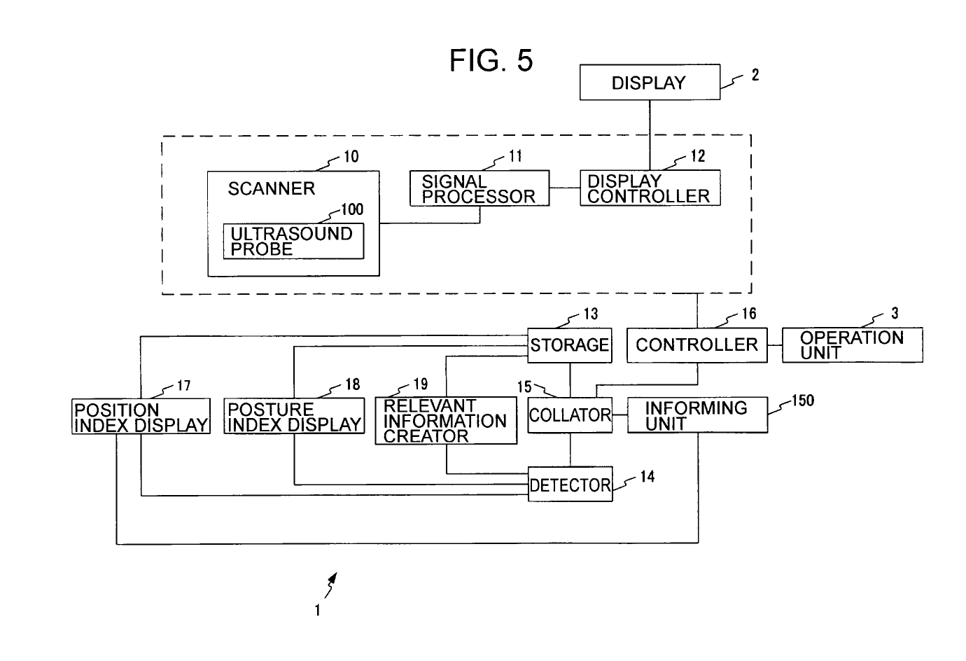

FIG. 5 is a block diagram representing the configuration of an ultrasound diagnosis apparatus 1 of the second embodiment. The ultrasound diagnosis apparatus 1 of the embodiment comprises the storage 13, the detector 14, the collator 15, and the controller 16, each forming the different configuration from the ones in the first embodiment. The ultrasound diagnosis apparatus 1 of the embodiment further comprises a relevant information creator 19 in addition to what the ultrasound diagnosis apparatus comprises in the first embodiment. The other configuration of the ultrasound diagnosis apparatus 1 of the embodiment is the same as the one in the first embodiment.

### (Storage 13)

The storage 13 previously stores first somatotype information representing the somatotype of the subject P in the past test. The storage 13, for example, stores a plurality of position information of characteristic points of the abdominal girth of the subject P in the past test. Also, the first somatotype information may include the contour information representing the contours of the body surface of the subject P in the past test.

### (Detector 14)

The detector 14 detects second somatotype information representing the somatotype of the subject P. The detector 14, for example, detects the plurality of position information of characteristic points of the abdominal girth of the subject P. The second somatotype information may also include the contour information representing the contours of the body surface of the subject P. The detector 14 may detects the positional information of the characteristic points using the same technique used in the first embodiment.

### (Collator 15)

The collator 15 collates the first somatotype information with the second somatotype information. The collator 15, for example, detects the characteristic points corresponding to the characteristic points represented in the first somatotype information from the second somatotype information, and calculates the positional difference (distance and direction) between the characteristic points represented in the first somatotype information and the detected characteristic points in the second somatotype information.

### (Relevant Information Creator 19)

The relevant information creator 19 creates third relevant information in which a third relative position corresponding to the first relative position for the second somatotype information is related to the operation content, based on the collation result resulted from the collator 15 and the first relevant information. The relevant information creator 19 calculates the third relative position which should be related to the operation content represented in the first relevant information in the first embodiment, based on the difference calculated by the collator 15 and the first relative position. The relevant information creator 19 relates the calculated third relative position with the operation content represented in the first relevant information to create the third relevant information.

### (Controller 16)

In the collation by the collator 15, when the second relative position detected by the detector 14 and the third relative position are matched, the controller 16 controls at least one of the scanner 10, the signal processor 11, and the display controller 12, based on the operation content related to the third relative position.

### Effect

Effect of the ultrasound diagnosis apparatus 1 of the embodiment is described.

In the ultrasound diagnosis apparatus 1 of the embodiment, the storage 13 previously stores the first somatotype information representing the somatotype of the subject P in the past test. The detector 14 detects the second somatotype information representing the somatotype of the subject P. The collator 15 collates the first somatotype information with the second somatotype information. The relevant information creator 19 creates the third relevant information in which the third relative position corresponding to the first relative position for the second somatotype information is related to the operation content, based on the collation result resulted from the collator 15 and the first relevant information. When the second relative position and the third relative position are matched in the collation by the collator 15, the controller 16 controls at least one of the scanner 10, the signal processor 11, and the display controller 12, based on the operation content related to the third relative position. In this way, even when testing the subject P whose somatotype is different from what he was like in the past test, the ultrasound diagnosis apparatus 1 can test the subject P according to the same operation content as the one in the past test. Thereby, even when testing the subject P whose somatotype is different from what he was like in the past test, it is possible to provide an ultrasound diagnosis apparatus which can easily and precisely recreate the same test conditions as the one in the past.

### Third Embodiment

An ultrasound diagnosis apparatus of a third embodiment is an ultrasound diagnosis apparatus which can read out the operation content in the past tests from an external storage, and test according to the same operation content.

### Configuration

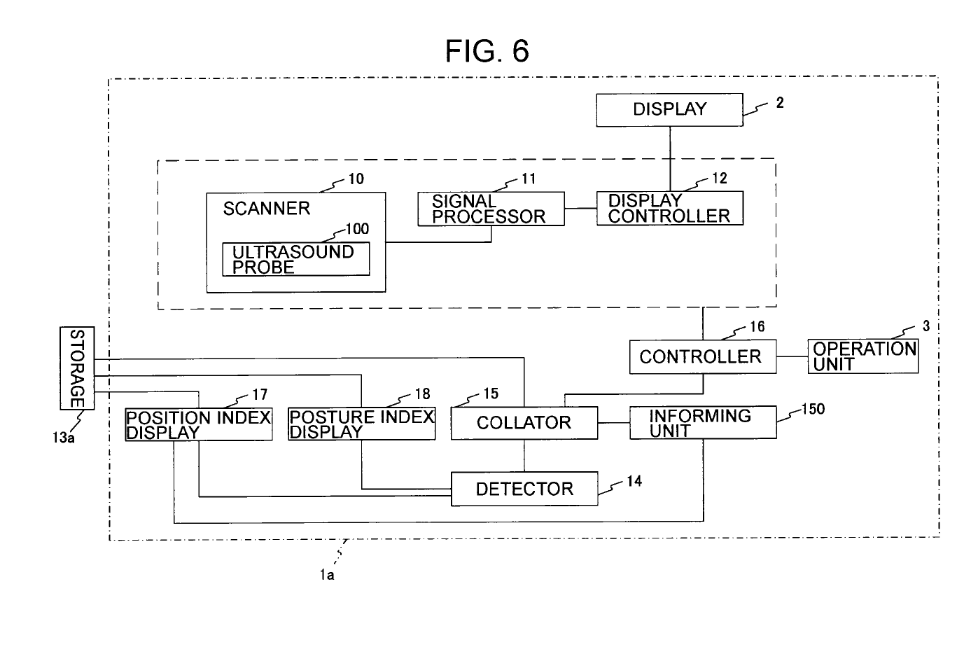

FIG. 6 is a block diagram representing the configuration of an ultrasound diagnosis apparatus 1 a of the third embodiment. The ultrasound diagnosis apparatus la of the embodiment reads out the relative position and the operation content in the past test from a storage 13a externally arranged in the apparatus. The ultrasound diagnosis apparatus 1a and the storage 13a may be connected through a general communication interface. Hereinafter, the descriptions of the same items as in the first embodiment may be omitted.

The storage 13a relates the operation content of at least one of the scanner 10, the signal processor 11, and the display controller 12 in a past test to the relative position of the ultrasound probe 100 for the subject P in the past test to store in advance. The storage 13a may also store the posture information representing the posture of the subject P in the past test. The past test includes the past test performed by the ultrasound diagnosis apparatus 1a, or the past test performed by an ultrasound diagnosis apparatus different from the ultrasound diagnosis apparatus 1a, or both of those.

The scanner 10 scans the subject P with ultrasound waves through the ultrasound probe 100. The signal processor 11 implements the signal processing on the signals from the scanner 10. The display controller 12 causes the display 2 to display images based on the output from the signal processor 11. The detector 14 detects the relative position of the ultrasound probe 100 for the subject P in the present test.

The collator 15 collates the relative position detected by the detector 14 with the relative position in the past test. At this time, the collator 15 receives the relative position in the present test from the detector 14, and reads out the relative position in the past test from the storage 13a. In this collation, the collator 15 outputs whether the relative position in the present test is matched with the relative position in the past test or not as the collation result. Further, when the relative position in the present test and the relative position in the past test are matched, the collator 15 reads out the operation content related to the relative position in the past test from the storage 13a to output the content to the controller 16.

The controller 16 controls at least one of the scanner 10, the signal processor 11, and the display controller 12, based on the operation content of the relative position in the past test, when the relative position detected by the detector 14 is matched with the relative position in the past test in the collation by the collator 15. That is, during the test, when the ultrasound probe 100 is positioned at the same relative position as the one in the past test, the operation content related to the relative position (that is, the operation content in the past test) is recreated by the controller 16.

Based on the relative position of the ultrasound probe 100 for the subject P in the past test previously stored in the storage 13a, the position index display 17 displays the position index representing the relative position. The posture index display 18 displays the posture index imitating the posture of the subject P in the past test, based on the posture information stored in the storage 13a.

### Effect

Effect of the ultrasound diagnosis apparatus 1a of the embodiment is described.

The ultrasound diagnosis apparatus 1a of the present embodiment comprises the scanner 10, the signal processor 11, the display controller 12, the detector 14, the collator 15, and the controller 16. The scanner 10 scans the subject P with ultrasound waves through the ultrasound probe 100. The signal processor 11 implements the signal processing on the output from the scanner 10. The display controller 12 causes the display 2 to display images based on the output from the signal processor 11. The detector 14 detects the relative position of the ultrasound probe 100 for the subject P in the present test. The collator 15 collates the relative position detected by the detector 14 with the relative position in the past test. In the collation by the collator 15, when the relative position detected by the detector 14 is matched with the relative position in the past test, the controller 16 controls at least one of the scanner 10, the signal processor 11, and the display controller 12, based on the operation content of the relative position in the past test. Further, the storage 13a is externally arranged in the ultrasound diagnosis apparatus la, and relates the operation content of at least one of the scanner 10, the signal processor 11, and the display controller 12 in the past test with the relative position of the ultrasound probe 100 for the subject P in the same past test to store in advance. In this way, the ultrasound diagnosis apparatus la can test by recreating the operation content of the past test, when the ultrasound probe 100 is positioned at the same position as the one in the past test. Thereby, it is possible to provide an ultrasound diagnosis apparatus which can easily recreate the same test conditions as in the past.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

### EXPLANATION OF SYMBOLS

1 1a Ultrasound diagnosis apparatus
2 Display
3 Operation unit
10 Scanner
11 Signal processor
12 Display controller
13 13a Storage
14 Detector
15 Collator
16 Controller
17 Position index display
18 Posture index display
19 Relevant information creator
100 Ultrasound probe
150 Informing unit
IP IPi Position index
P Subject

1. An ultrasound diagnosis apparatus comprising:
a scanner configured to scan a subject with ultrasound waves through an ultrasound probe;
a signal processor configured to implement signal processing on the signals from the scanner;
a display controller configured to cause a display to display images based on the output from the signal processor;
a storage configured to previously store first relevant information in which operation content of at least one of the scanner, the signal processor, and the display controller in a past test is related to a first relative position which is the relative position of the ultrasound probe for the subject in the past test;
a detector configured to detect a second relative position which is the relative position of the ultrasound probe for the subject in the present test;
a collator configured to collate the first relative position represented in the first relevant information and the second relative position detected by the detector; and
a controller configured to control at least one of the scanner, the signal processor, and the display controller based on the operation content related to the first relative position, when the first relative position and the second relative position are matched in the collation by the collator.

2. The ultrasound diagnosis apparatus according to claim 1, wherein the storage is configured to previously store the first relevant information in which at least one of scanning conditions of the ultrasound waves from the scanner, signal processing conditions for the signal processor, and display conditions of the images for the display controller are included as the operation content.

3. The ultrasound diagnosis apparatus according to claim 1, wherein
in the first relevant information previously stored in the storage, the first relative position includes relative positions of plural parts of the ultrasound probe for the subject;
the detector is configured to detect the relative positions of the plural parts of the ultrasound probe for the subject as the second relative position; and
the collator is configured to collate the first relative positions and the second relative positions for the plural parts of the ultrasound probe.

4. The ultrasound diagnosis apparatus according to claim 1, further comprising a position index display configured to display a position index representing the first relative position, based on the first relevant information.

5. The ultrasound diagnosis apparatus according to claim 4, wherein the position index display has a transparent medium and is configured to display the position index on a part of the medium, and be wearable by an operator.

6. The ultrasound diagnosis apparatus according to claim 4, wherein
the storage is configured to previously store second relevant information in which order information representing the moving order of the ultrasound probe in a past test is related to the first relative position , and
the position index display is configured to display a plurality of position indexes based on the first relevant information and the second relevant information.

7. The ultrasound diagnosis apparatus according to claim 1, wherein
the storage is configured to previously store posture information representing the posture of the subject in the past test, and
further comprises a posture index display configured to display a posture index imitating the posture of the subject in the past test, based on the posture information.

8. The ultrasound diagnosis apparatus according to claim 7, wherein
the posture index display has a transparent medium, and is configured to display the posture index on a part of the medium, and be wearable by the operator.

9. The ultrasound diagnosis apparatus according to claim 7, wherein the posture information includes contour information representing the contours of the body surface of the subject.

10. The ultrasound diagnosis apparatus according to claim 1, wherein
the storage is configured to previously store first somatotype information representing the somatotype of the subject in the past test;
the detector is configured to detect second somatotype information representing the somatotype of the subject;
the collator is configured to collate the first somatotype information and the second somatotype information;
the collator further comprises a relevant information creator configured to create third relevant information in which third relative position corresponding to the first relative position for the second somatotype information is related to the operation content, based on the collation result resulted from the collator and the first relevant information;
the collator is configured to collate the second relative position and the third relative position, and
the controller is configured to control at least one of the scanner, the signal processor, and the display controller, based on the operation content related to the third relative position, when the second relative position and the third relative position are matched in the collation by the collator.

11. The ultrasound diagnosis apparatus according to claim 10, wherein,
the posture information includes contour information representing the contours of the body surface of the subject.

12. The ultrasound diagnosis apparatus according to claim 1, further comprising,
an informing unit configured to inform the collation result resulted from the collator.

13. An ultrasound diagnosis apparatus comprising:
a scanner configured to scan a subject with ultrasound waves through an ultrasound probe;
a signal processor configured to implement signal processing on the signals from the scanner;
a display controller configured to cause a display to display images based on the output from the signal processor; and
a storage configured to previously store first relevant information in which operation content of at least one of the scanner, the signal processor, and the display controller in a past test is related to a first relative position which is the relative position of the ultrasound probe for the subject in the past test.

14. The ultrasound diagnosis apparatus according to claim 13, wherein the storage is configured to previously store the first relevant information in which at least one of scanning conditions of the ultrasound waves from the scanner, signal processing conditions for the signal processor, and display conditions of the images for the display controller are included as the operation content.

15. The ultrasound diagnosis apparatus according to claim 13, further comprising a position index display configured to display a position index representing the first relative position, based on the first relevant information.

16. The ultrasound diagnosis apparatus according to claim 15, wherein
the storage is configured to previously store second relevant information in which order information representing the moving order of the ultrasound probe in the past test is related to the first relative position , and
the position index display is configured to display a plurality of position indexes based on the first relevant information and the second relevant information.

17. The ultrasound diagnosis apparatus according to claim 13, wherein
the storage is configured to previously store posture information representing the posture of the subject in the past test, and
further comprises a posture index display configured to display a posture index imitating the posture of the subject in the past test, based on the posture information.

18. An ultrasound diagnosis apparatus comprising:
a scanner configured to scan a subject with ultrasound waves through an ultrasound probe;
a signal processor configured to implement signal processing on the signals from the scanner;
a display controller configured to cause a display to display images based on the output from the signal processor;
a detector configured to detect a relative position of the ultrasound probe for the subject in the present test;
a collator configured to collate the relative position detected by the detector and the relative position in a past test; and
a controller configured to control at least one of the scanner, the signal processor, and the display controller based on the operation content of the relative position in the past test, when the relative position detected by the detector and the relative position in the past test are matched in the collation by the collator.
